(19) United States
(12) Patent Application Publication
(10) Pub. No.: US 2009/0054334 A1
(43) Pub. Date: Feb. 26, 2009

(54) Combinatorial improvement of bifunctional drug properties

(76) Inventors: Mitchell W. Mutz, Mountain View, CA (US); Jason E. Gestwicki, Ann Arbor, MI (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/154,596

(22) Filed: May 23, 2008

Related U.S. Application Data
(60) Provisional application No. 60/931,390, filed on May 23, 2007.

(51) Int. Cl. A61K 38/16; A61K 31/4523

(52) U.S. Cl. 514/12; 514/320

(57) ABSTRACT

A method is provided for improving at least one pharmacokinetic property and maintaining or improving affinity of a therapeutic upon administration to a host. In the method, one administers to the host an effective amount of a bifunctional compound of less than about 5000 Daltons comprising the therapeutic or an active derivative, fragment or analog thereof and a recruiter ligand moiety. The recruiter ligand moiety binds to at least one biomoiety. The bifunctional compound has at least one modulated pharmacokinetic property upon administration to the host and equivalent or greater affinity for a target of the therapeutic as compared to a free drug control that comprises the therapeutic. In addition, the overall drug efficacy is improved by the steric bulk of the bifunctional complexed with the recruited biomoiety.

### CROSS-REFERENCE TO RELATED APPLICATIONS

This application claims priority to U.S. provisional application No. 60/931,390, filed May 23, 2007, which is incorporated by reference in its entirety.

### TECHNICAL FIELD

This invention relates generally to pharmacology and more specifically to the modification of known active agents to give them more desirable properties.

### BACKGROUND

Bifunctional drug compounds have achieved success in the drug market. Traditional, monofunctional, drugs suffer from a host of potential problems related to drug toxicity, non-specificity, toxicity of dose formulation, poor tissue targeting, and suboptimal pharmacokinetics. One common example of a class of bifunctional drug has been protein-conjugated drug molecules, for example albumin covalently attached to paclitaxel (sold as Abraxane). Abraxane has a higher maximum tolerated dose than the parent, monofunctional paclitaxel. However, this approach, while conferring advantages over the monofunctional drug, still suffers from the disadvantage of requiring a relatively expensive protein formulation ($4000 per dose for Abraxane) and suboptimal efficacy. A different approach to leveraging the advantages of a biomoiety-conjugated drug in a small (less than 5000 Dalton bifunctional drug) has been disclosed (Briesewitz, U.S. Pat. Nos. 6,887,842, 6,921,531, and 6,372,712). In these disclosures, the bifunctional compound consists of a drug compound covalently attached to a recruiter ligand. The recruiter ligand typically binds non-covalently to a biomoiety that improves a drug property relative to the monofunctional drug compound. Improved properties disclosed previously may include one of efficacy or pharmacokinetics. The improvement in efficacy may be achieved by changes in pharmacokinetics or affinity of the bifunctional drug to the target. The general bifunctional strategy is to improve drug properties by binding to a non-target protein via a ligand covalently attached to the drug moiety and termed the recruiter ligand herein. The recruiter ligand binds to a recruited biomoiety which may often be a protein. The non-target protein provides steric bulk to shield the drug molecule from hepatic clearance and increase the circulating half-life. However, the prior art has not provided solutions to some major challenges in this bifunctional approach: decreased oral bioavailability due to large molecular weights, poor solubility, lower target binding due to steric hindrance caused by the biomoiety, unknown effects of linkers used to attach drugs to the bifunctional moiety, balancing the competing equilibria and kinetics of drug target binding and recruiter binding to the non-target biomoiety, and overcoming xenobiotic pumping mechanisms. Additionally, the bifunctional approach biases intra-vs. extracellular bifunctional drug distribution, and this property must also be optimized for different drug classes. In particular, several ligands disclosed for extracellular protein binding in the prior art such as warfarin are not advisable due to the risk of uncontrolled bleeding posed by warfarin. In short, the benefits of a bifunctional approach using a recruiter ligand are best realized by the parallel optimization of drug properties not considered in the prior art and single property approaches are unlikely to yield a best-in-class pharmaceutical.

The prior art of bifunctional optimization has focused on a single property optimization approach, focusing mainly on pharmacokinetics (PK) or affinity. The optimization of multiple properties is generally more complex than the single property approach. However, to compete with best-in-class drugs, the optimization of multiple drug properties (for example, solubility, pk, affinity, oral bioavailability, association constants and off rate constants of drug and ligand) is highly essential. For example, a protease inhibitor-cyclosporine bifunctional conjugate has been prepared (M. Solomon, thesis, University of Wisconsin, 1998). This drug exhibited reasonable efficacy in a cell infectivity assay but had an ED₅₀ that was merely comparable to the parent, monofunctional protease inhibitor (also referred to herein as free drug). Although the immunosuppressive functionality of the cyclosporin moiety was supposedly eliminated, the drug data does not create a compelling case to replace existing protease inhibitors. Moreover, due to the increased expense of the bifunctional vs. monofunctional drug, being comparable in efficacy is inadequate for an improved drug. In another example, a bifunctional paclitaxel conjugated to the 2′ OH position of paclitaxel has been prepared using a known ligand for FKBP protein, SLF, or synthetic ligand for FKBP, created by Dennis Holt. While comparable ED₅₀ was achieved for this bifunctional in an in vitro cell assay, improved solubility and improved formulation was not seen for this compound. Although prior art suggested that the SLF ligand would indeed yield a compound with better efficacy either through better drug binding affinity or pk, the data showed that these bifunctional drug properties require further refinement, either by a better FKBP ligand design, use of a different recruited biomoiety, optimized solubility, or other properties. Since albumin-conjugated paclitaxel has provided a lower-toxicity formulation of paclitaxel, a compound that requires Cremaphor, a known toxic drug vehicle, is undesirable in any improved paclitaxel bifunctional and would be unlikely to provide a substantial improvement over existing drugs.

In the case of a synthesis of an improved bifunctional protease inhibitor, a low yield of a bifunctional synthesis was seen related to the charge distribution and linker length in the initial design. The short linker contributed to a low synthetic yield due to steric hindrance of the ligand and drug moiety and the proximity of like charge groups also hindered the synthesis. The potential benefit of the short linker (improved oral bioavailability due to the lower molecular weight of the bifunctional) was offset by the low yield of the synthesis. Avoiding close proximity of like moieties in a bifunctional synthesis presents additional complications of bifunctional design.

An additional benefit to patients of a bifunctional approach is the simultaneous optimization of both pharmacokinetics and affinity. This presents a technical challenge since properties such as enhanced steric bulk of the bifunctional bound to a recruited biomoiety may shield a drug from enzymatic degradation, but the same steric bulk of the recruited biomoiety may also prevent a bifunctional drug from interacting with the active site of a target compound (Wandless). In such a case, the benefit of the improved pk may be offset by the decreased affinity of the bifunctional compound relative to the parent (monofunctional) compound. Judicious target selection does permit simultaneous affinity and pk optimization. For example, a protease inhibitor moiety was chosen that destabilizes the HIV protease two-helix bundle, In this case, the additional steric bulk of the bifunctional complexed with the recruited biomoiety does provide simultaneous affinity and pk optimization. To leverage the advantage of steric bulk, judicious drug target choice is required. Moreover, the improvement of these properties must be achieved while maintaining good drug solubility and preferably allowing improved formulations.

There is therefore still a need in the art for drugs and associated dosage forms that have reduced first pass clearance and/or improved pharmacokinetics, while being relatively economical to produce, and are still effective in maintaining affinity for the drug target. Ideally, these drugs associate with a non-target protein to take advantage of steric bulk, but the non-covalent association must also allow the drugs to bind effectively to the target as well. Additionally, these bifunctional drugs should be orally bioavailable when necessary. Moreover, drugs that are antibiotics or chemotherapeutics should optimally counteract xenobiotic pumping mechanisms via association with the recruited biomoiety to provide additional therapeutic benefits of bifunctional drugs relative to the parent compound.

### SUMMARY OF THE INVENTION

In an embodiment of this invention, a method for modulating multiple properties of a bifunctional therapeutic upon administration to a host is provided. One administers to the host an effective amount of a bifunctional compound of less than about 5000 Daltons comprising the therapeutic or an active derivative thereof and a recruiter ligand. The recruiter ligand binds to at least one intracellular biomoiety. The biomoiety is commonly a protein but may also be a nucleic acid, lipid, carbohydrate, or other biological component. The bifunctional compound has a plurality of modulated properties upon administration to the host as compared to a free drug control and are one or more of the following improved properties: solubility, efficacy, synthetic yield, organ targeting, oral bioavailability, optimized intra vs. extracellular distribution, and optimized equilibrium binding constants of drug and recruiter ligand, resistance to xenobiotic pumps, and enhanced affinity.

In a further embodiment of this invention, novel recruiter ligands are employed to achieve improved bifunctional drug properties relative to a monofunctional control.

In a further aspect of the invention, a bifunctional compound is provided in a pharmaceutical formulation that sustains the ability of the compound to cross cell membranes and avoid catalysis by cytochrome p450 enzymes and other drug-degrading catalysts inside cells.

In a further aspect of the invention, biasing the drug to remain inside cells increases efficacy by a two-fold mechanism: avoiding extracellular Cytp450 enzymes and avoiding intracellular degradation by enzymes via an association with a non-target intracellular protein which confers protection from intracellular enzymes. The non-target protein must still allow binding to the drug target and optimally enhances the binding affinity measured directly by the association constant, Ka, or enhances efficacy. The bifunctional drug is chosen in indications where enhanced steric bulk helps improve drug affinity and efficacy.

In a further aspect, the bifunctional drug has lower toxicity than the parent compound because a lower dose is required to achieve equivalent efficacy due to enhanced concentration/hour (area under the curve) and that non-target binding is directed to a high abundance, non-target protein (albumin, HSP90, FKBP12, etc.). Also, the recruiter ligand design is used to enhance the solubility of the bifunctional drug relative to the parent compound.

In a further aspect of the invention, the bifunctional drug is particularly effective in reducing the size of drug resistant tumors since the enhanced binding to the non-target protein has a lower equilibrium dissociation constant or dissociation rate constant than the dissociation constant or dissociation rate constant of the monofunctional compound with protein complexes that pump drugs and other xenobiotics out of cells such as the MDR or multi-drug resistant protein family found in both prokaryotic and eukaryotic cells.

### FIGURES

FIG. 1 depicts the structure of SLF linked to a modular linker and target binding moiety, for example an anticancer therapeutic. Due to the modular nature of the synthesis, the linker group and target-binding group may be readily altered.

FIG. 2 illustrates how the steric bulk of a protein can confer protection from enzymes.

In FIG. 3, the left side depicts the bimodal binding character of FK506 whereby it binds both FKBP and calcineurin. The schematic on the right depicts how the calcineurin-binding mode can be eliminated by substituting a linker and target binding moiety. In this manner, FK506 can simultaneously target FKBP and bind a second protein. Synthetic ligands with no affinity for calcineurin such as SLF may also be used.

In FIG. 4A, we see the structure of FK506 bound to curcumin. FIG. 4B illustrates how FK506-curcumin is protected from CYP3a4, a P450 enzyme, in the presence of FKBP. FIG. 4C gives a schematic of the Invitrogen assay used.

In FIG. 5, the left side illustrates sample linkers that could be employed in a modular synthetic scheme.

FIG. 6 exhibits a synthetic scheme for a bifunctional form of paclitaxel. See S. Wang et al., Bioorg. Med. Chem. Lett., 16, 2628-2631 (2006).

FIG. 7 illustrates the efficacy of a bifunctional paclitaxel drug in cell culture. It provides an example of enhanced efficacy of bifunctional paclitaxel in the absence of drug-degrading enzymes. The lower o.d. indicates more tumor cell growth inhibition by paclitaxel-SLF (right bar in each pair).

FIG. 8 shows the difference in partitioning between extra- and intracellular space due to the presence of the recruiter ligand moiety in an in vivo mouse model study.

FIG. 9 shows the effect of area under the curve for a bifunctional compound in mice vs. a monofunctional compound. Compound was administered via a tail vein injection to mimic intravenous drug administration. The data shows a 25 fold increase in area under the curve for the bifunctional vs. the monofunctional.

FIG. 10 shows the efficacy of the paclitaxel bifunctional in a xenograft tumor mouse model vs. a vehicle control containing the Cremaphor-ethanol solvent only.

### DETAILED DESCRIPTION OF PREFERRED EMBODIMENTS

Before describing the present invention in detail, it is to be understood that this invention is not limited to specific solvents, materials, or device structures, as such may vary. It is also to be understood that the terminology used herein is for the purpose of describing particular embodiments only, and is not intended to be limiting.

As used in this specification and the appended claims, the singular forms “a,” “an,” and “the” include both singular and plural referents unless the context clearly dictates otherwise. Thus, for example, reference to “an active ingredient” includes a plurality of active ingredients as well as a single active ingredient, reference to “a temperature” includes a plurality of temperatures as well as single temperature, and the like.

The term “bifunctional compound” refers to a non-naturally occurring compound that includes a recruiter ligand moiety and a drug moiety, where these two components may be covalently bonded to each other either directly or through a linking group. The term “drug” refers to any active agent that affects any biological process. Bifunctional compounds may have more than two functionalities.

The recruiter ligand moiety may be a peptide or protein and may also be an enzyme or nucleic acid. Similarly, the drug moiety may also be peptide, protein, enzyme, or nucleic acid.

Active agents which are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g. cell death, cell proliferation etc.

By “pharmacokinetic property” is meant a parameter that describes the disposition of an active agent in an organism or host. Representative pharmacokinetic properties include: drug half-life, hepatic first-pass metabolism, volume of distribution, degree of blood serum protein, e.g. albumin, binding, etc, degree of tissue targeting, cell type targeting.

By “half-life” is meant the time for one-half of an administered drug to be eliminated through biological processes, e.g. metabolism, excretion, etc.

By “hepatic first-pass metabolism” is meant the propensity of a drug to be metabolized upon first contact with the liver, i.e. during its first pass through the liver.

By “volume of distribution” is meant the distribution and degree of retention of a drug throughout the various compartments of an organism, e.g. intracellular and extracellular spaces, tissues and organs, etc.

The term “efficacy” refers herein to the effectiveness of a particular active agent for its intended purpose, i.e. the ability of a given active agent to cause its desired pharmacologic effect. A functional test may also be applied to determine the ED₅₀, the concentration at which 50% of cell growth is inhibited, or the concentration at which 50% of a binding event is inhibited. In the case of an enzyme inhibitor, enzyme activity as a function drug concentration can also be used.

The term “in vivo efficacy” is used to denote testing in an organism (prokaryotic or eukaryotic) since a primary feature of the bifunctionals discussed herein is the ability to escape enzymatic degradation and defeat drug efflux mechanisms in a biological context. It is often the case that the efficacy of the bifunctional as defined by ED₅₀ in an in vitro context will not differ greatly from the free drug control in a simple binding or even a cell assay. However, the “in vivo efficacy” can vary greatly from in vitro efficacy since the bifunctional and free drug control are now challenged with drug-degrading enzymes, xenobiotic pumping mechanisms, the burden of correct intra- vs. extra-cellular distribution, and distribution in the recipient host. Relative in vivo efficacy is normally assessed using free drug control and bifunctional in series of concentrations where molarity of free drug control vs. molarity of the bifunctional are varied across substantially similar range in a prokarytoic or eukaryotic organism.

The term affinity refers to the binding constant of two moieties with units of molar. In the bifunctional case, the binding constant can be for the bifunctional drug with the drug target. It may also be measured for the recruiter ligand and recruited biomoiety or any two moieties.

The term “host” refers to any mammal or mammalian cell culture or prokaryotic cell.

Where the term cancer is used, it is understood that the invention may be employed on relative chemotherapeutics such as found in other any type of cancer including those cancers found in non-human species or human variants.

Where the term “intracellular” protein is used, this includes any protein created intracellularly in a cell and then may optionally be extruded to the extracellular space or reside in the cell membrane or remain inside the cell.

The term “metronomic therapy” refers to long-term preventive anti-cancer chemotherapy where a drug is administered over a much longer term (many months or years instead of weeks) to avoid recurrence of tumors. Normally, toxicity dictates the use of chemotherapy at very low doses that compromise the effectiveness of this mode of therapy.

The term “biomoiety” refers to a protein, DNA, RNA, ligand, carbohydrate, lipid, or any other component molecule of a prokaryotic or eukaryotic organism.

The term “recruited protein” refers to the non-drug target protein bound by the ligand.

The term “recruited biomoiety” refers to the non-drug target moiety bound by the ligand which may be a protein, nucleic acid, lipid, carbohydrate, or other biological entity.

The term “koff rate” refers to the timescale (seconds, minutes, hours) wherein the bifunctional is released from its binding partner. The koff rate constant is a first order constant in units of s⁻¹.

For the binding reaction A+B [symbol] [AB], “K_{D}” is described by [A][B]/[AB], with corresponding kinetic rate constants of d[A]/dt=d[B]/dt=k_{off}[AB] and d[AB]/dt=kₒₙ[A][B]. At equilibrium, d[AB]/dt=d[A]/dt=d[B]/dt, and therefore, k_{off}/kₒₙ=[A][B]/[AB]=K_{D}. K_{D} is further known as an equilibrium dissociation constant.

The term “affinity” refers to the K_{D} and describes the concentration at which substantial at which two moieties exhibit substantial binding (vide supra).

The term “recruiter ligand” refers to a molecule that binds to a biomoiety that is different from the drug target bound by the drug in a bifunctional molecule.

The recruiter ligand is attached to the drug with a linker that may contain between 0 and 100 atoms.

Pharmacokinetic modulating moiety is often synonymous with recruiter ligand moiety but recruiter ligand is often preferred since the presence of this ligand alters a plurality of properties of the bifunctional drug such as bioavailability, efficacy, binding constant, solubility, and toxicity, among others, and many of these are not pharmacokinetic properties.

The term “plurality” herein indicates one or more.

The term “attached” may indicate covalent or non-covalent attachment. The attachment of the recruiter ligand for the recruited biomoiety is generally non-covalent.

The term “drug efflux mechanism” may refer to a naturally occurring prokaryotic or eukaryotic pump which has the tendency to remove a foreign chemical from a biological context. The mammalian p-glycoprotein pump complex tends to inhibit adsorption of xenobiotic compounds from the gut into the circulation. Tumor cells have related xednobiotic pumps which contribute to drug resistance. Bacteria are also equipped with mechanisms to remove non-native substances from bacterial cells.

Where FK506 is used, variants or analogs of FK506 are included, such as rapamycin, pimecrolimus, or synthetic ligands of FK506 binding proteins (SLFs) such as those disclosed in U.S. Pat. Nos. 5,665,774, 5,622,970, 5,516,797, 5,614,547, and 5,403,833 or described by Holt et al., “Structure-Activity Studies of Synthetic FKBP Ligands as Peptidyl-Prolyl Isomerase Inhibitors,” Bioorganic and Medicinal Chemistry Letters, 4(2):315-320 (1994). Small FKBP ligands have been described (U.S. Pat. No. 5,614,547 and 6509477).

In an embodiment of this invention, a method for modulating at least one pharmacokinetic property and lowering toxicity of a therapeutic upon administration to a host is provided. One administers to the host an effective amount of a bifunctional compound of less than about 5000 Daltons comprising the therapeutic or an active derivative thereof and a recruiter ligand moiety. The recruiter ligand moiety binds to at least one intracellular protein. The bifunctional compound has at least one modulated pharmacokinetic property upon administration to the host as compared to a free drug control that comprises the therapeutic and has lower toxicity as compared to a free drug control that comprises the therapeutic.

Another embodiment of the invention is maintaining efficacy of the bifunctional since adding chemical bulk to the free drug and the association of a recruited biomoiety as well as linker can substantially reduce drug moiety to drug target binding. Moreover, adding chemical bulk normally reduces the oral bioavailability of a drug according to the Christopher Lipinsky “rule of 5.”

Bifunctional compound in general have aroused considerable interest in recent years. See, for example, U.S. Pat. Nos. 6,270,957, U.S. Pat. No. 6,316,405, U.S. Pat. No. 6,372,712, U.S. Pat. No. 6,887,842, and U.S. Pat. No. 6,921,531. ConjuChem (Montreal, Canada) scientists have shown that covalent coupling of insulin to human serum albumin can improve the half-life from 8 hours to over 48 hours. Xenoport (Santa Clara, Calif.) has pioneered attachment of receptor ligands to improve drug uptake and distribution. Human trials of methotrexate-albumin conjugates revealed that the albumin conjugated methotrexate had half-lives of up to two weeks compared with 6 hours for unmodified methotrexate. Other examples include PEGylation of growth factors and attachment of folate groups that “target” anti-cancer drugs. All these strategies use modification of a “parent” drug to provide new binding profiles or enhanced protection from degradation.

However, the albumin-conjugated drugs raise the expense of drug production and make it highly improbable for compounds to diffuse in and out of cells.

More recently, a team including one of the inventors attached SLF to ligands for amyloid beta. Amyloid beta oligomers are believed to underlie the neuropathology of Alzheimer's disease. Therefore, methods to decrease amyloid aggregation are of therapeutic interest. Amyloid ligands, such as congo red or curcumin (above), can be synthetically coupled to FK506 or SLF. The resulting bifunctional compound binds both FKBP and amyloid beta. These molecules are potent inhibitors of amyloid aggregation and they block neurotoxicity in cell culture. See Jason E. Gestwicki et al., “Harnessing Chaperones to Generate Small-Molecule Inhibitors of Amyloid β Aggregation,” Science 306:865-69 (2004). FKBP ligands which improve the ability of a drug to cross the blood-brain barrier are also of interest.

Bifunctional compounds of the type employed in the present invention are generally described by the formula:

X-L-Z

wherein:

X is a drug moiety;

L is a bond or linking group; and

Z is a recruiter ligand moiety or may be termed a recruiter ligand since a plurality of properties may be changed

(pharmacokinetics, efficacy, intra and extracellular distribution, toxicity, k_{off} rates, solubility, oral bioavailability, etc.)
with the proviso that X and Z are different. Thus, as may be seen, a bifunctional compound is a non-naturally occurring or synthetic compound that is a conjugate of a drug or derivative thereof and a recruiter ligand moiety, where these two moieties are optionally joined by a linking group.

In bifunctional compounds used in the invention the pharmacokinetic modulating and drug moieties may be different, such that the bifunctional compound may be viewed as a heterodimeric compound produced by the joining of two different moieties. In many embodiments, the recruiter ligand moiety and the drug moiety are chosen such that the corresponding drug target and any binding partner of the recruiter ligand moiety, e.g., a pharmacokinetic modulating protein to which the recruiter ligand moiety binds, do not naturally associate with each other to produce a biological effect.

The bifunctional compounds are typically small. As such, the molecular weight of the bifunctional compound is generally at least about 100 D, usually at least about 400 D and more usually at least about 500 D. The molecular weight may be less than about 800 D, about 1000 D, about 1200 D, or about 1500 D, and may be as great as 2000 D or greater, but usually does not exceed about 5000 D. The preference for small molecules is based in part on the desire to facilitate oral administration of the bifunctional compound. Molecules that are orally administrable tend to be small.

The recruiter ligand moiety modulates a pharmacokinetic property, e.g. half-life, hepatic first-pass metabolism, volume of distribution, degree of albumin binding, etc., upon administration to a host as compared to free drug control. By modulated pharmacokinetic property is meant that the bifunctional compound exhibits a change with respect to at least one pharmacokinetic property as compared to a free drug control. For example, a bifunctional compound of the subject invention may exhibit a modulated, e.g. longer, half-life than its corresponding free drug control. Similarly, a bifunctional compound may exhibit a reduced propensity to be eliminated or metabolized upon its first pass through the liver as compared to a free drug control. Likewise, a given bifunctional compound may exhibit a different volume of distribution that its corresponding free drug control, e.g. a higher amount of the bifunctional compound may be found in the intracellular space as compared to a corresponding free drug control. Analogously, a given bifunctional compound may exhibit a modulated degree of albumin binding such that the drug moiety's activity is not as reduced, if at all, upon binding to albumin as compared to its corresponding free drug control. In evaluating whether a given bifunctional compound has at least one modulated pharmacokinetic property, as described above, the pharmacokinetic parameter of interest is typically assessed at a time at least 1 week, usually at least 3 days and more usually at least 1 day following administration, but preferably within about 6 hours and more preferably within about 1 hour following administration.

The linker L, if not simply a bond, may be any of a variety of moieties chosen so that they do not have an adverse effect on the desired operation of the two functionalities of the molecule and also chosen to have an appropriate length and flexibility. The linker may, for example, have the form F₁—CH₂)ₙ—F₂ where F₁ and F₂ are suitable functionalities. A linker of this sort comprising an alkylene group of sufficient length may allow, for example, for the free rotation of the drug moiety even when the recruiter ligand moiety is bound. Alternatively, a stiffer linker with less free rotation may be desired. The hydrophobicity or hydrophobicity of the linker is also a relevant consideration. FIG. 5 depicts some precursors which may be used for the linker (with the carboxyl functionality protected). For the current strategy, the linker also needs to permit the simultaneous binding of recruited biomoiety and drug target. Additionally, the linker must allow substantial oral bioavailability (at least 10%).

The drug moiety X may, in certain embodiments of the invention, preferably be an anticancer therapeutic. The drug moiety may be derived from a known anticancer therapeutic, which is preferably effective against one or more types of cancer. The drug moiety preferably has a functionality which may readily and controllably be made to react with a linker precursor. The known chemotherapeutics are generally susceptible to metabolism and subsequent deactivation by hepatic first-pass or subsequent pass clearance mechanisms. Cancer chemotherapy is an active area of research. Other embodiments may include any type of drug class, however. Further embodiments may also include analgesics, anti-inflammatory, and anti-infective drugs.

Certain of the concepts of this invention have applicability to other drug moieties besides chemotherapeutics. In general, bifunctional compounds may usefully be made with any drug having a suitable moiety capable of reacting with linkers and which has a need for pharmacokinetic modulation as well as properties of solubility, efficacy, and lower toxicity. Thus, for example, drugs having a strong first-pass effect may be candidates for incorporation into a bifunctional compound.

In general, the recruiter ligand moiety Z will be one which is capable of reversible attachment to a common protein, meaning one which is abundant in the body or in particular compartments of the body or particular tissue types. Common proteins include, for example, FK506 binding proteins, cyclophilin, tubulin, actin, heat shock proteins, and albumin. Common proteins are present in concentrations of at least 1 micromolar, preferably at least 10 micromolar, more preferably at least 100 micromolar, and even more preferably 1 millimolar in the body or in particular compartments or tissue types. The recruiter ligand moiety should, like the drug, have a moiety which is capable of reacting with suitable linkers.

It is desirable for at least some embodiments of the present invention that the binding of the recruiter ligand moiety Z to a common protein be such as to sterically hinder the activity of common metabolic enzymes such as CYP450 enzymes when the bifunctional compound is so bound. Persons of skill in the art will recognize that the effectiveness of this steric hindrance depends, among other factors, on the conformation of the common protein in the vicinity of the recruiter ligand moiety's binding site on the protein, as well as on the size and flexibility of the linker. The choice of a suitable linker and recruiter ligand moiety may be made empirically or it may be made by means of molecular modeling of some sort if an adequate model of the interaction of candidate pharmacokinetic modulating moieties with the corresponding common proteins exists. The linker choice must balance parameters of length, hydrophobicity, attachment point to the drug target, and attachment point to the ligand.

The attachment point and linker characteristics are preferably selected based on structural information such that the inhibitory potency of the therapeutic is preserved, giving the desired superior pharmacokinetic characteristics.

Where the recruiter ligand moiety operates by binding a protein, it may be referred to as a “presenter protein ligand” and the protein which it binds to may be referred to as a “presenter protein.” Where this moiety modulates a plurality of properties and not just pharmacokinetics, this moiety is called a recruiter ligand. In a preferred embodiment, multiple bifunctional drug properties are improved relative to a monofunctional drug to yield a substantially improved bifunctional drug.

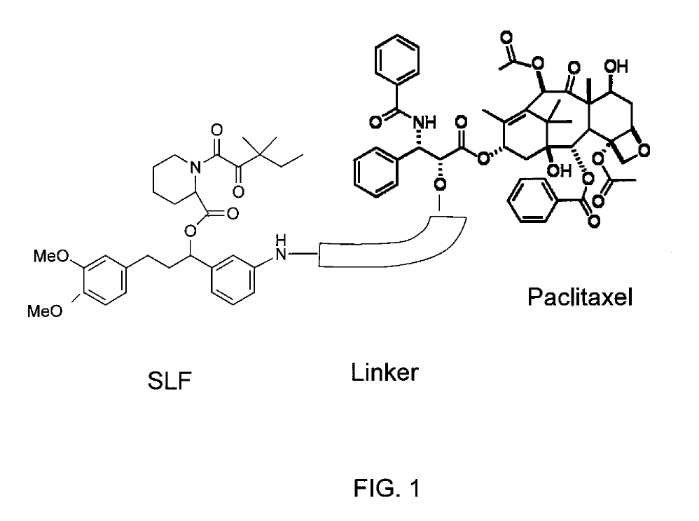

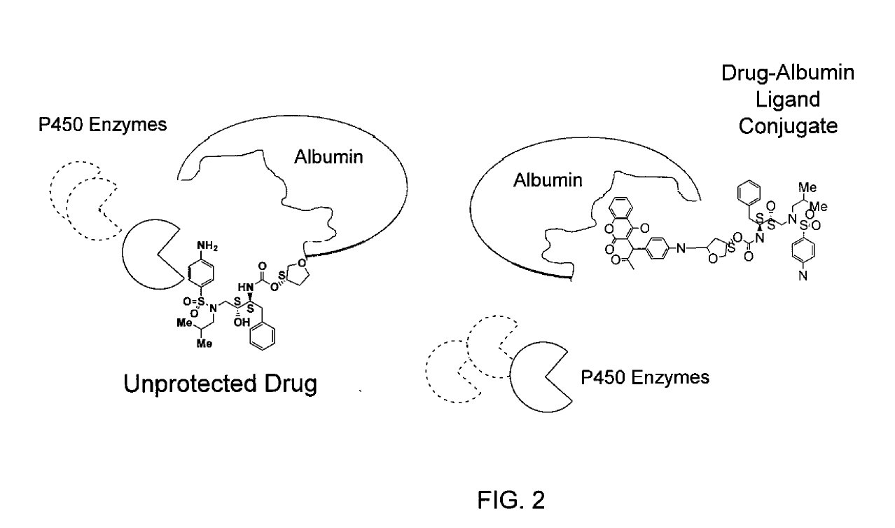

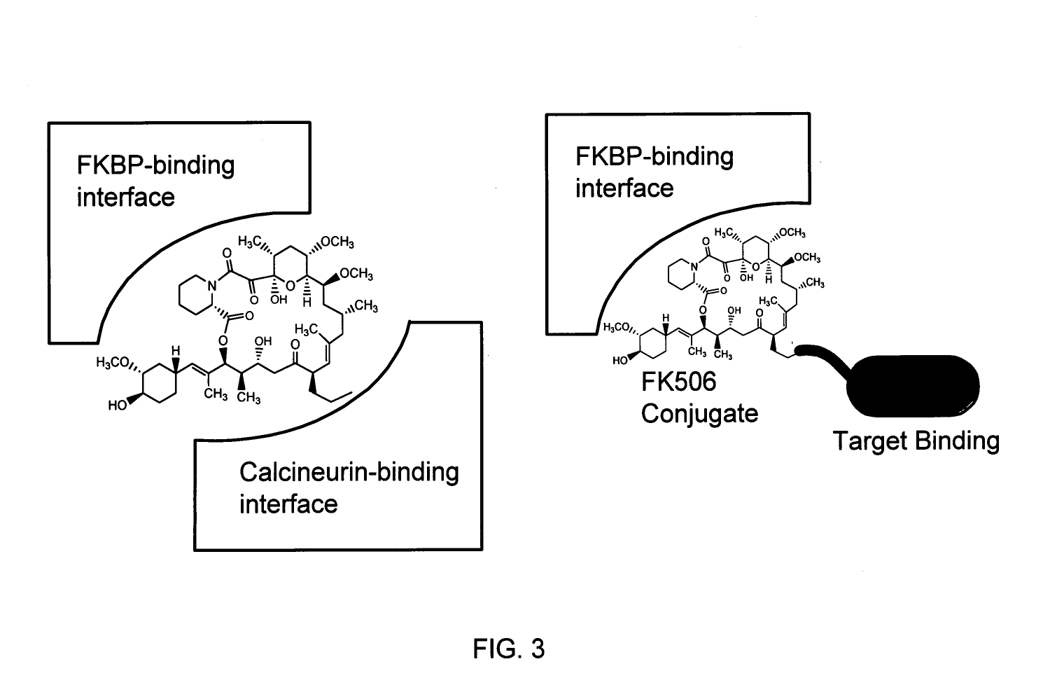

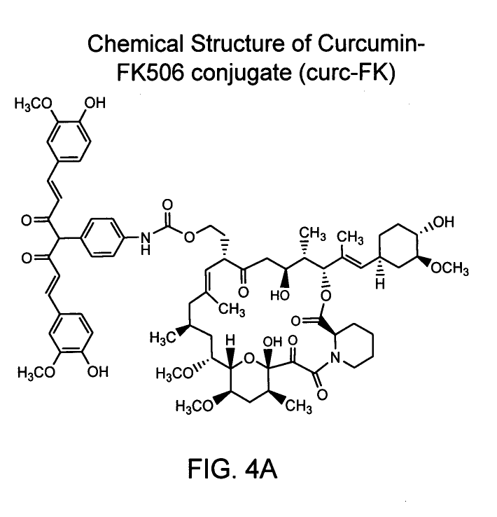

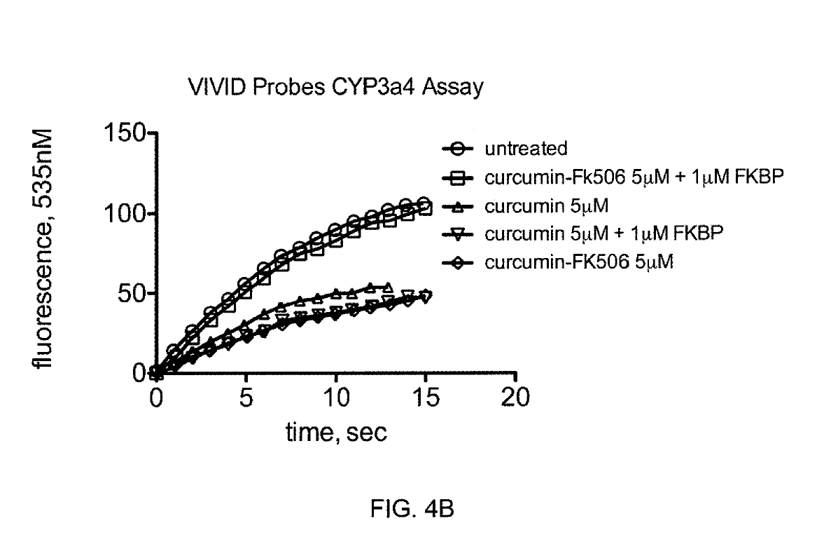

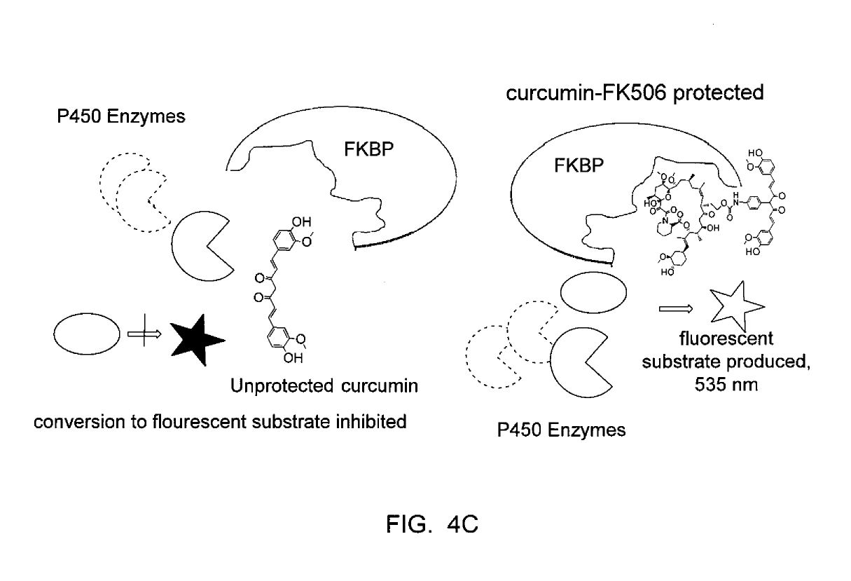

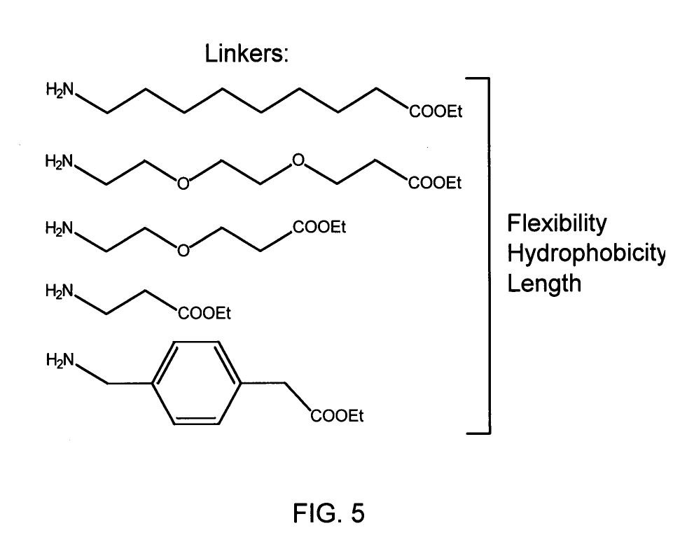

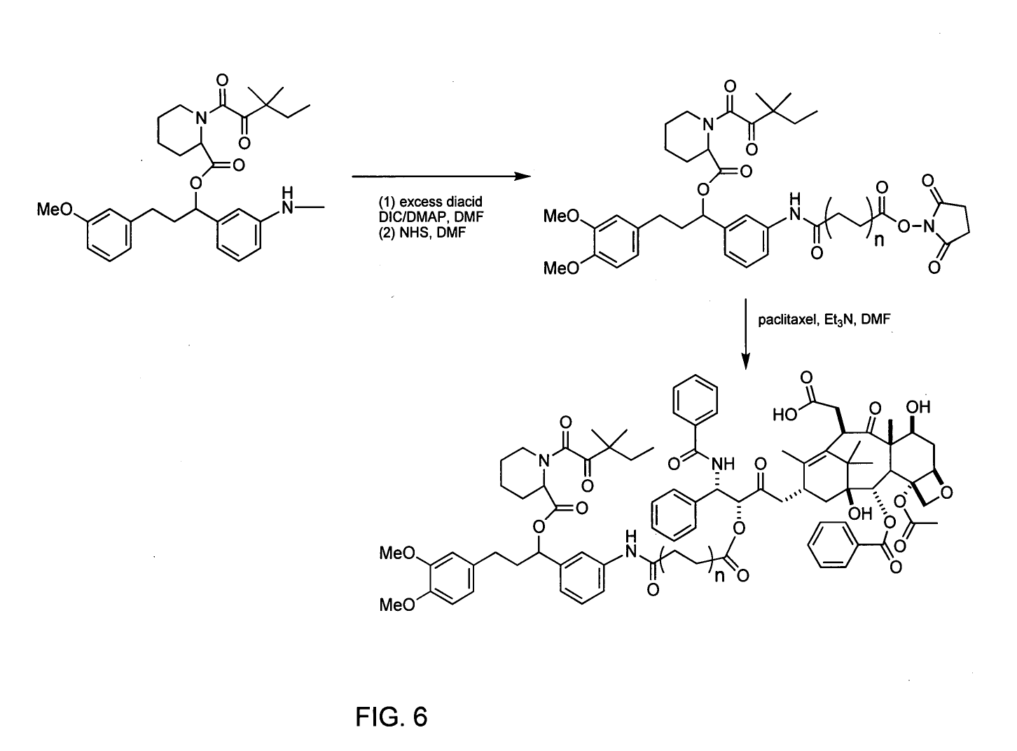

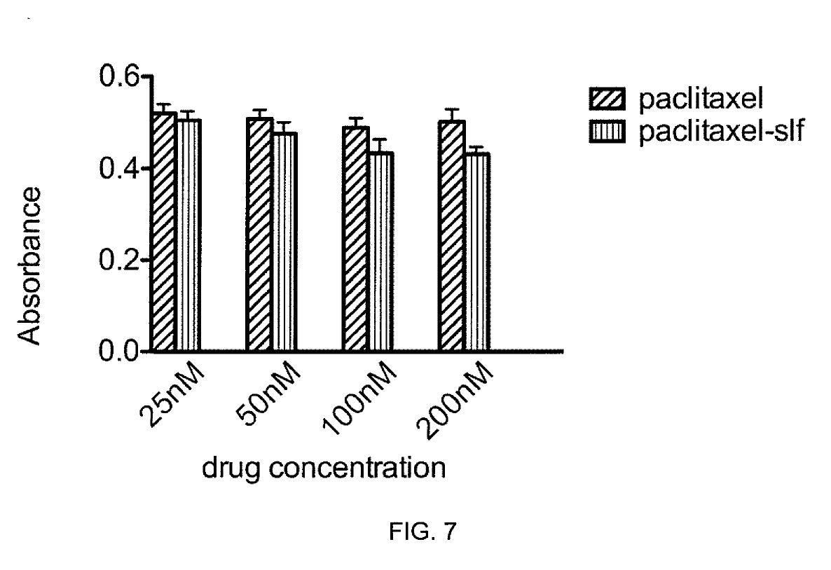

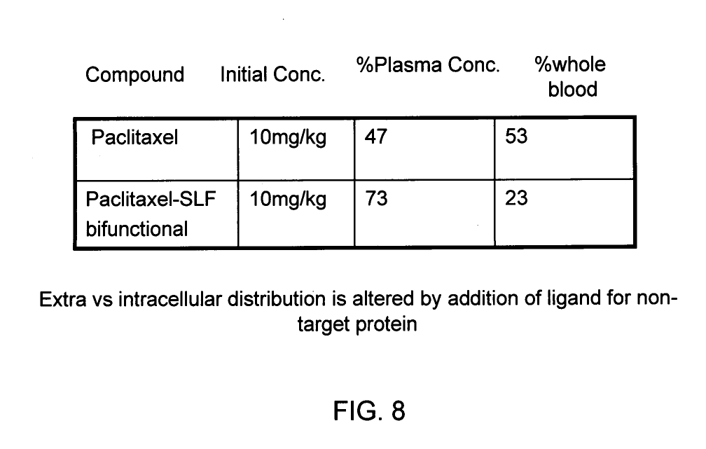

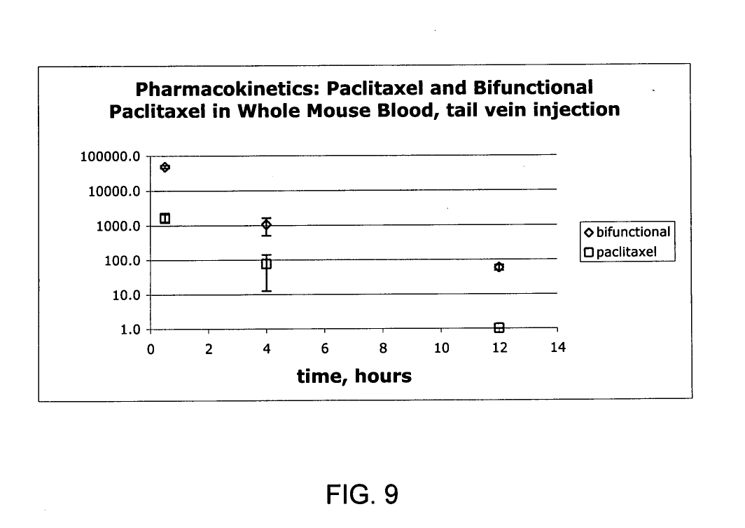

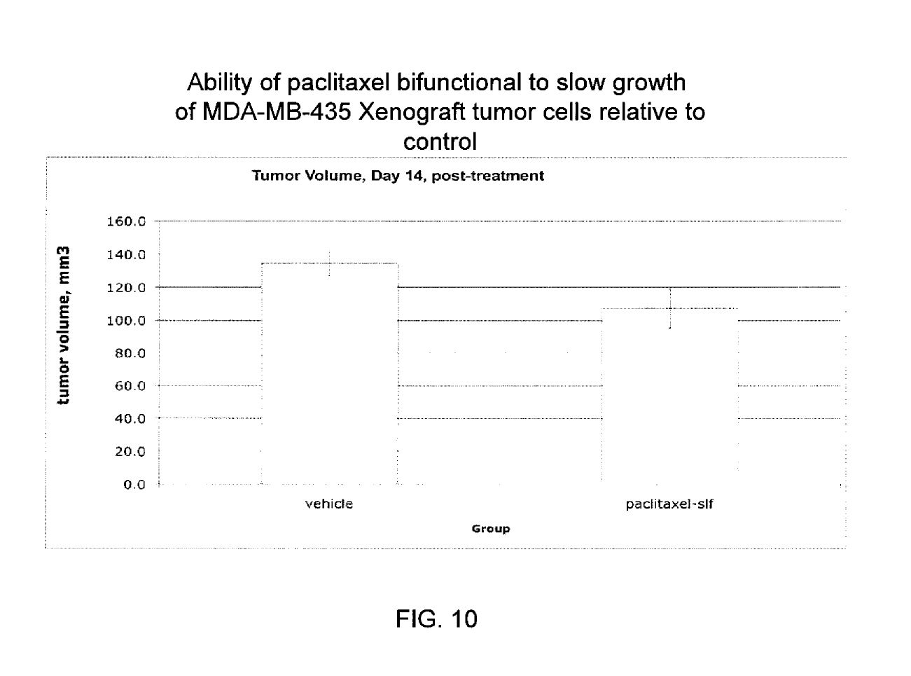

The recruiter ligand moiety may be, for example, a derivative of FK506, which has high affinity for the FK506-binding protein (FKBP), as depicted for example in FIG. 1. There are many synthetic ligands for FKBP. The abundance of FKBP (millimolar) in blood compartments, such as red blood cells and lymphocytes, makes it likely that a significant proportion of a dose of bifunctional compounds comprising FK506 would partition into blood cells and would dynamically equilibrate between the intracellular and extracellular space. A mechanism that tends to increase the portion of the chemotherapeutic dose that winds up in red blood cells and CD4+ lymphocytes will have a favorable effect on anti-cancer activity, as these sites are prime targets of chemotherapy. The steric bulk conferred by FKBP would hinder an anticancer therapeutic moiety from fitting into the binding pocket of intracellular enzymes (aldolases, hydroxylases, etc.) and so would prevent degradation via this class of enzymes. However, the steric bulk of the drug must not compromise drug target binding or bias the drug away from the preferred target organ.

An inactive form of FK506 may be preferable in some applications to avoid the possibility of side effects due to the possible interaction of the active FK506-FKBP complex with calcineurin. It may be advantageous to use FKBP binding molecules such as synthetic ligands for FKBP (SLFs) described by Holt et al., supra. This class of molecule is lower molecular weight than FK506, and that is generally advantageous for drug delivery and pharmacokinetics. Additionally, SLF has no binding affinity for calcineurin and cannot suppress immune function. For illustrative purposes, some diagrams will show examples of the use of FK506, though it should be understood that the same strategy can apply to other ligands of peptidyl prolyl isomerases such as the FKBP proteins and that ligands for other presenter proteins may be employed.

The value of FK506 and other FKBP binding moieties as pharmacokinetic modulating moieties of the invention is further supported by the following. FK506 (tacrolimus) is an FDA-approved immunosuppressant. It has been determined that FK506 can be readily modified such that it loses all immunomodulatory activity but retains high affinity for FKBP. FKBP is an abundant chaperone that is particularly prevalent (˜ millimolar) in red blood cells (rbcs) and lymphocytes. The complex between FK506-FKBP gains affinity for calcineurin and inactivation of calcineurin blocks lymphocyte activation and causes immunosuppression.

This interesting mechanism of action is derived from FK506's chemical structure. FK506 is bifunctional; it has two non-overlapping protein-binding faces. One side binds FKBP, while the other binds calcineurin. This property provides FK506 with remarkable specificity and potency. Moreover, FK506 has a long half-life in non-transplant patients (21 hrs) and excellent pharmacological profile. In part, this is because FK506 is unavailable to metabolic enzymes via its high affinity for FKBP, which favors distribution into protected cellular compartments (72-98% in the blood). It can be expected that suitable bifunctional compounds with an FKBP-binding recruiter ligand moiety will likewise possess some favorable characteristics of inactive FK506, namely, good pharmacokinetics and blood cell distribution. Importantly, lower molecular weight ligands for FKBP may be used to allow improved pharmacokinetics or oral bioavailability. FK506 has a mass of 804 daltons and has a lower bioavailability than smaller FKBP ligands.

In general, the recruiter ligand moiety will have a molecular weight less than about 2000 D, less than about 1800 D, less than about 1500 D, less than about 1100 D, less than about 900 D, less than about 500 D, or less than about 300 D.

It is also possible to co-administer the biomoiety to which the recruiter ligand binds with the bifunctional compound in order to modify the pharmacokinetics, toxicity, efficacy, and other properties to a greater degree than would be possible with just the native concentration of the common protein.

In a further embodiment of this invention, a method is provided for synthesizing a bifunctional compound comprising anticancer therapeutic functionality and the ability to bind to a common protein.

The synthesis of the bifunctional compound starts with a choice of suitable recruiter ligand and drug moiety. It is desirable to identify on each of these moieties a suitable attachment point which will not result in a loss of biological function for either one. This is preferably done based on the existing knowledge of what modifications result or do not result in a biological function. On that basis, it may reliably be conjectured that certain attachment points on the pharmacokinetic and drug moieties do not affect biological function. Likewise, in FIG. 6 one sees a secondary amine functions on SLF, which can serve as an attachment point to the 2′ hydroxyl moiety on paclitaxel.

A general synthetic strategy is to locate a secondary amine on the drug moiety at which the drug moiety can be split (so that the secondary amine does not form part of any cycle in the drug moiety). The secondary amine is chosen such that, from experimental or other considerations, it is believed that the drug will retain its efficacy if only the portion of the drug moiety to one side of the secondary amine is present. The portion of the drug moiety to that side of the secondary amine is then synthesized by any appropriate technique, with the secondary amine in the synthesized molecule being protected during synthesis by an appropriate protecting moiety such as Boc. The protecting moiety is then removed, leaving a primary amine which may react with a carboxyl group through a variety of known chemistries for making a peptide bond (see, e.g., J. Mann et al., Natural Products. Their Chemistry and Biological Significance (1994), chapter 3). FIG. 6 gives an example of this synthetic strategy.

In a further aspect of the invention, a bifunctional compound comprising a therapeutic moiety is formulated, for example in the form of a tablet, capsule, or parenteral formulation, to make a pharmaceutical preparation. The pharmaceutical preparation may be employed in a method of treating a patient having cancer against which the anticancer therapeutic moiety is effective. For example, if the anticancer therapeutic moiety is effective against breast cancer, the pharmaceutical preparation may be administered to a patient suffering from breast cancer.

For the preparation of a pharmaceutical formulation containing bifunctional compounds as described in this application, a number of well known techniques may be employed as described for example in Remington: The Science and Practice of Pharmacy, Nineteenth Ed. (Easton, Pa.: Mack Publishing Company, 1995).

In a further aspect of the invention, a bifunctional compound with a plurality of improved properties relative to the free drug is formulated as part of a controlled release formulation in which an additional controlled release mechanism besides the effect of the recruiter ligand moiety is employed to achieve desirable release characteristics. The bifunctional compound is as above, comprising a drug moiety, a linker, and a recruiter ligand moiety. In this aspect of the invention, a drug moiety may be an anticancer therapeutic or a different type of drug.

Drugs which are candidates for bifunctionalization followed by application of other controlled release technologies may belong to a wide variety of therapeutic categories including, but not limited to: analeptic agents; analgesic agents; anesthetic agents; anti-arthritic agents; respiratory drugs, including anti-asthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; anti-inflammatory agents; antimigraine preparations; antinauseants; antiparkinsonism drugs; antipruritics; antipsychotics; antipyretics; antispasmodics; antitubercular agents; anti-ulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral.

Exemplary drugs presently known to have high first-pass metabolism include HIV chemotherapeutics as discussed above, paclitaxel, methotrexate, vinblastine, verapamil, morphine, lidocaine, acebutolol, isoproterenol, and desipramine. The formation of bifunctional compounds is particularly appropriate for these drugs.

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXPERIMENTAL

The general method for combinatorial optimization of bifunctional drug compounds is to apply a set of kinetic and physiochemical selection criteria that are particularly beneficial to this class of bifunctional compound. The assessment of drug binding to the target must be balanced against the potential tendency of a ligand with a bulky biomoiety attached to interfere with target binding. Moreover, the affinity of ligand for the non-target protein must be designed so that the bifunctional is not resident inside cells for too long a time. For example, if the time required for the dissociation of a bifunctional compound from FKBP was hours, bifunctional compound would likely be excessively trapped inside a target cell so that new cells resulting from mitosis would lack bifunctional compound. Therefore, unlike conventional drug chemistries, the ligand-non-target protein dissociation constant should be in the nanomolar regime, for applications such as chemotherapeutics where the drug binding constant is often also in the nanomolar regime. This is contrary to current drug optimizations where binding affinities are typically optimized to be as low as possible (i.e. picomolar preferred over nanomolar) to allow efficacy at lower doses. Off-rate measurements of bifunctional molecules from the drug target can be complicated by the need to assess the off rate in the context of a recruited biomoiety. When possible, the non-target biomoiety is added exogenously, since the present of a binding partner for the drug and recruiter ligand will result in the most accurate kinetic analysis.

An additional consideration with bifunctional compounds has to do with the poorly characterized role of the linker connecting drug and ligand moieties. Linkers that are too short preclude simultaneous drug-target interaction and ligand-non-target protein interaction. However, linkers that excessively increase the weight of the bifunctional and are likely to decrease adsorption and bioavailability of orally administered therapeutics. The linker should also not interfere with compound solubility or may be used to enhance the solubility of the bifunctional drug.

Ligand choice and design also play a role in oral bioavailability, particularly in the context of uptake from the small intestine. Traditional ligands for FKBP, albumin, and other high-abundance proteins may exceed 500, 600 or even 1000 daltons. The large size is normally a disadvantage for

oral absorption, synthetic yield, and cost of production. Therefore, the design of relatively compact, soluble ligands with appropriate affinities for recruited biomoieties is also lacking in the current art.

A further consideration for bifunctional design is the need for the recruited biomoiety to be included in many of the more traditional screening processes. For example, in a CaCo-2 cell line model of the uptake of compounds for bioavailability, the addition of the ligand target is important for accurate analysis of the diffusion of the compound through monolayers of CaCo-2 cells in the case of an extracellular ligand that can affect partitioning. In the intracellular ligand case, Caco-2 cells would naturally harbor ligand targets such as FKBP proteins.

This consideration of adding the recruited biomoiety also pertains to screening methods to assess if the bifunctional drug diffuses across the blood-brain barrier. In this case, a method using co-cultures of endothelial cells and organotypic brain slices may be employed as an in vitro screen for bifunctionals and recruiter ligand library compounds which diffuse across the blood brain barrier as described by S. Duport et al. Proc. Natl. Acad. Sci. USA Vol. 95, pp. 1840-1845, February 1998.

### Example 1

### Optimizing Linker Length and Kinetics

The test compound, a bifunctional, protease inhibitor-SLF conjugate, is made with the following linker unit chemistries: glycol, alkene, imine. The number of subunits is varied as follows: 1,3,5,7,9 subunits in length connecting inhibitor and SLF moieties. The synthetic yield is calculated. Next efficacy of the bifunctional drug target is measured by an in vitro cell infectivity assay and binding to the drug target is made via surface plasmon resonance on a Biacore. The k_{off} rate constant is further analyzed by Biacore to determine if the off rate is in the regime of several seconds to minutes, In this manner. linker length is examined by kinetic analysis to provide a mechanistic basis for differences in drug residence time in cells and to optimize efficacy. Linker chemistry may also be used to optimize oral bioavailability and solubility.

### Example 2

### Optimizing Off Rates and Adsorption from the Gut

A Caco-2 cell line is used as a model for adsorption from the gut, Compounds with good permeability are expected to have superior oral bioavailability. In this Caco-2 protocol, the flux of the test article from the apical to the basolateral side of Caco-2 cells is evaluated in order to predict the absorption of compounds from the lumen of the intestine. Since this is a HTS protocol, only a single concentration of the test article and a single incubation time will be used in order to accommodate a large number of test articles. A typical protocol is discussed below:

1. Dissolve each bifunctional article in an appropriate solvent (e.g., DMSO) to prepare a 100× stock solution (e.g., 5 mM). Dilute this stock solution in Apical Transport Buffer to prepare a 1× dosing solution (e.g., 50 μM). Many researchers choose a standard dosing concentration of 50 μM and N=1 to 3 replicate wells for experiments in which they will only be screening absorption at a single concentration. In addition, DMSO may be used as a diluent with a final concentration no higher than 5% in the final apical dosing solution without significantly interfering with the absorption of most compounds.

2. Add 0.1 mL of each 1× test article dosing solution to the apical side of individual Transwells. Gently pipette the solution onto the surface so as not to disrupt the delicate cell monolayer. Replace the plate cover and place the 24-well plate on a shaker (if an orbital shaker is used, set it to 50 to 60 rpm) inside a 37° C., 5% CO₂ incubator with saturating humidity. The use of a shaker is optional, but is recommended to eliminate the “unstirred layer” phenomenon that is likely to occur if no shaker is used.

3. Incubate the plates for 2 hours. Remove the plates from the incubator. Analyze the concentration of the test article present in the Basolateral Transport Buffer below each Transwell and in the medium used to dose each Transwell. Typical analysis is by HPLC, LC/MS, or liquid scintillation counting, as appropriate. For purposes of calculating flux rates or percent absorption, analysis of the dosing solutions is preferred over the media remaining on the apical side of the Transwells. This is because non-specific binding of the test article to the Caco-2 cells and/or the Transwells may lead to artifacts in the data.

4. Compounds with acceptable permeability (2-% or higher) can be subjected to a kon/koff rate analysis via surface plasmon resonance on a BiaCore (Pharmacia).

Preferred dissociation rates of bifunctional from recruited biomoiety are on the order of milliseconds to several minutes to allow the bifunctional compound to diffuse to newly created cells and not be trapped for too long in a given cell. For many small molecules, the second order rate constant is diffusion limited and is approximated with a maximum value of 10⁹ M⁻¹s⁻¹. For the binding reaction A+B [symbol] [AB], K_{D} is described by [A][B]/[AB], with corresponding kinetic rate constants of d[A]/dt=d[B]/dt=k_{off}[AB] and d[AB]/dt=kₒₙ[A][B]. At equilibrium, d[AB]/dt=d[A]/dt=d[B]/dt, and therefore, k_{off}/kₒₙ=[A][B]/[AB]=K_{D}.

The relationship at equilibrium for the dissociation equilibrium constant, K_{D} and k_{off} and kₒₙ rate constants is therefore:

K_{D}=k_{off}/kₒₙ

For a drug compound with a nanomolar dissociation equilibrium constant, the k_{off} rate constant is on the order of 1 s⁻¹. So, working backwards, nanomolar binding constants are preferred for designed recruiter ligands for the desired k_{off} rate constants provided the drug equilibrium dissociation constant is also in the low nanomolar regime and the second order association rate constant of drug and drug target is on the order of 10⁹ M⁻¹s⁻¹. If nanomolar binding is not achieved for drug and drug target binding, the k_{off} rate constant must be adjusted to achieve the appropriate residence time in a cell. The rate constant measurements can be performed using standard commercial instrumentation such as a Biacore where off rate constants can be monitored by surface plasmon resonance in a label-free methodology.

### Examples 3

### Optimizing Extra and Intra-Cellular Distribution

The recruiter ligand choice is used to bias extra and intracellular distribution. The bias is dependent on the choice of drug target. Drugs such as insulin operate on extracellular receptors and there is no efficacy advantage to internalizing the protein to the intracellular space. However, many chemotherapeutics such as paclitaxel bind to an intracellular component such as tubulin, thus making an intracellular bias desirable. Nonetheless, overly biasing the distribution in the intracellular case will make it impossible for the drug to spread its effect over a large number of cells given the limited dose amount (typically 130 mg/kg in humans). Overbiasing the drug in the extracellular case will make it difficult to target the drug to specific locations if the therapeutic must cross cell membranes to achieve effective transport.

So, the tuning of the extracellular and intracellular distribution is important, and must be engineered using both the K_{D} and k_{off} parameters of the bifunctional binding to the drug target and bifunctional binding to the recruited biomoiety.

Once a decision is made for either an extra- or intra-cellular bias, the ligand is chosen accordingly, Moreover the ligand is designed to strike the correct balance to allow cell membrane transport where necessary. The bias may determined kinetically as described above by determining affinity and kinetic parameters for the bifunctional with respect to the drug target and recruited biomoiety. Kinetic and endpoint distributions in plasma and whole blood are determined by liquid chromatography and mass spectroscopy.

A typical protocol for the determination of compartmentalization into whole blood is as follows:

1. Add drug (10 mM in DMSO) to 250 μl whole blood for a final concentration of 10.0 & 30.0 μM
2. Incubate in shaker at 37° C. for 240 minutes
3. Separate plasma by centrifuging at 1,000×g (3706 rpm) for 15 minutes at 4.0° C. Plasma samples may be stored at −80° C. at this point.
4. Transfer 75 μl of plasma to new microfuge tubes
5. Extract drug by adding 1 ml ethyl acetate 2× and vortexing vigorously
6. Centrifuge at 12,000×g (13,200 rpm) for 7 minutes at 4.0° C.
7. Transfer supernatants to fresh glass vials
8. Evaporate to dryness using RotoVap
9. Reconstitute residue in 500 μl of acetonitrile/0.1% acetic acid
10. Inject onto LCMS

Plasma samples are run in the same manner to determine the ratio of compound in whole blood vs. plasma where the plasma sample represents the extra-cellular blood fraction and whole blood samples contain both the intra- and extra-cellularly distributed drug.

### Example 4

### Ligand Size and Solubility Optimization

Ligand size can greatly effect several drug properties: binding kinetics to the recruiter, oral bioavailability, cell membrane permeability, and bifunctional solubility, and extra- vs intracellular distribution, among others. Again a multi-component optimization is most productive in considering the design of an optimal ligand for a bifunctional drug application. One example of a ligand for FKBP protein is FK506. This drug has a half life of 11.3 hours and a bioavailability of 20%. The molecular weight of 804 daltons likely accounts for the low bioavailability since drugs with masses of over 500 daltons tend to have lower bioavailability than drugs with substantially higher molecular weights. For example, MGI Pharmaceutical's GPI-1485 is a ligand for FKBP12 with a molecular weight of less than 300 daltons and is 50% bioavailable. Intriguingly, this ligand also crosses the blood-brain barrier. For many applications such as the treatment of Alzheimer's disease or other neurological disorders, ligands which cross the blood-brain barrier and enhance efficacy are of high value These lower weight FKBP ligands (under 800 daltons) are therefore preferred in treating neurological disorders since the ligand properties will bias drugs towards better distribution into the brain when compared with larger FKBP ligands such as FK506 and other peptidyl prolyl isomerase ligands such as cyclosporin. GPI-1485 also has favorable solubility vs. the SLF ligand and therefore can improve the solubility of drugs requiring formulations which currently require toxic adjuvants for solvation and administration such as the use of Cremaphor in the administration of paclitaxel.

To optimize ligand size and solubility, structure/activity relationship modeling can be used to dock potential ligands for the desired recruiting biomoiety, followed by model permeability studies and pk studies to assess oral bioavailability and circulating half-life. In addition, protection against first pass hepatic clearance can also be studied in conjunction with these other properties using an in vitro cytochrome P450 assay. Again, unlike monofunctional drugs, paying careful attention to balancing drug binding kinetics and recruiter ligand binding kinetics and will help avoid sequestering the drug inside cells for too long a time period.

### Example 5

Oral Uptake Optimization/Defeat of Efflux Mechanism in Prokaryotic and Eukaryotic Cells

Both ligand and linker will require optimization to allow good oral bioavailability of bifunctional compounds. For example, paclitaxel bound to SLF using a linker with a total molecular weight of 1400 daltons was shown to have no oral bioavailability in a mouse model study. Reduction of ligand size, as well as adjusting the compound solubility is expected to have a favorable effect on oral bioavailabillity. Additionally, avoidance of p-glycoprotein-mediated pumping mechanisms will be helpful in increasing the bioavailability. At the same time, increasing the association of bifunctional drug with the recruited biomoiety will provide favorable competitive kinetics with xenobiotic efflux mechanisms. So, the bifunctional drug design requires additional design parameters compared with monofunctional drugs due to the ligand/recruiter interaction component. The extra burden for screening is offset by the benefit conferred by greater drug efficacy and lower toxicity. For example, an improved antibiotic may consist of a bifunctional with a recruiter moiety that inhibits the xenobiotic pumping mechanism of bacterial cells to remove or metabolize the drug moiety. In this case, the recruiter moiety must be optimized for this purpose: simple binding constant-based screens will not necessarily yield a recruiter that overcomes antibiotic drug resistance. Two screen for both oral uptake and defeat of efflux, a two part screen may be used where the following example is for an antibiotic.

Part I: Library Generation

1. A library of candidate FKBP ligands is screened. The library scaffold is based on the known binding site of FK506 on FKBP12 and compounds are generated by split and pool techniques and reactions are tracked in micro-reactors tagged with an radio frequency identification (RFID).

2. The library is screened using the Caco-2 cell line as described above or alternative methods (synthetic lipid).

3. Ligands with substantially enhanced Caco-2 permeability relative to FK506 are then selected and passed to parts II and III of the screen.

Part II: Screen for resistance to prokaryotic xenobiotic pump

1. Bacteria are grown on agar containing LB media and different concentrations of ampicillin as a control using a strain of bacteria that is not ampicillin resistant.

2. Ampicillin is covalently bound to FKBP ligands to form the ampicillin bifunctional molecules.

3. The same strain of bacteria is grown on agar containing LB media and different concentrations of ampicillin bifunctional. (screening may take place in well plates with agar due to the large number of samples instead of petri dishes)

4. Wells which inhibit bacterial growth at the lowest level of bifunctional compound concentration are selected as having a bifunctional with the greatest efficacy per mole of compound and should have a lower risk of drug toxicity.

Part III: Direct Competitive Binding Assay of Drug with Efflux Pump Complex

5. An additional screen can be performed using protein G sepharose beads and an IgG antibody conjugated to components of the multi-drug-resistant efflux pump (MDR) such as tolC⁻. Bifunctional drug candidates are incubated with the beads overnight, and then recombinant FKB12 is added to the mixture for another 12 hours, reactions taking place in 384 well plates. The presence of FKB12 should compete with binding of the bifunctional to the tolC⁻ gene product and increase the concentration of free bifunctional.

6. After spinning the plates, supernatant is assessed for the presence of bifunctional drug, relative to the bead fraction using HPLC analysis. Samples. with the highest drug concentration in the supernatant have shown superior ability of FKBP to compete with tolC⁻ binding and are good candidates for drugs which would be resistant to bacterial MDR complexes.

### Example 6

### Screening for Drug Efficacy Caused by Association of a Drug with Recruited Biomoiety Due to Increased Steric Bulk of Bifunctional with Recruited Biomoiety

The bifunctional drug moiety is first tested in an in vitro assay for efficacy. Optimally, the recruited biomoiety of the bifunctional as defined herein (vide supra) is added to the assay buffer in various concentrations. As the concentration is increased and ED₅₀ curves are plotted, the increased presence of biomoiety correlates with improvements in drug efficacy. Additionally, the efficacy of the bifunctional drug is assessed at different concentrations while keeping the concentrations of the bifunctional molecule constant.

We claim:

1. A method for improving at least one pharmacokinetic property and maintaining or improving affinity of a therapeutic upon administration to a host, the method comprising: administering to the host an effective amount of a bifunctional compound of less than about 5000 Daltons comprising the therapeutic or an active derivative, fragment or analog thereof and a recruiter ligand moiety,
wherein the recruiter ligand moiety binds to at least one biomoiety,
wherein the bifunctional compound has at least one modulated pharmacokinetic property upon administration to the host and equivalent or greater affinity for a target of the therapeutic as compared to a free drug control that comprises the therapeutic, and
wherein the overall drug efficacy is improved by the steric bulk of the bifunctional complexed with the recruited biomoiety.

2. The method according to claim 1, wherein the pharmacokinetic property is selected from the group consisting of half-life, hepatic first-pass metabolism, volume of distribution, and degree of blood protein binding.

3. The method according to claim 1, wherein the bifunctional compound is administered as a pharmaceutical preparation.

4. The method according to claim 1, wherein the host is a mammal.

5. The method according to claim 1 where the recruiter ligand moiety has a mass of less than 1100 Daltons and binds to a peptidyl prolyl isomerase.

6. The method according to claim 1 where the linker length calculated by adding average inter-atomic distances along the shortest covalent chain between drug moiety and recruiter moiety is at least 2.4 Å.

7. The method according to claim 1 where the linker length calculated by adding average inter-atomic distances along the shortest covalent chain between drug moiety and recruiter moiety is at least 4.8 Å.

8. The method according to claim 1 where the linker length calculated by adding average inter-atomic distances along the shortest covalent chain between drug moiety and recruiter moiety is at least 6.0 Å.

9. The method according to claim 1 where the linker length calculated by adding average inter-atomic distances along the shortest covalent chain between drug moiety and recruiter moiety is at least 7.2 Å.

10. The method according to claim 1 where the linker length calculated by adding average inter-atomic distances along the shortest covalent chain between drug moiety and recruiter moiety is at least 9.6 Å.

11. The method according to claim 1, wherein the partitioning of the bifunctional compound between the extracellular and intracellular space improves pharmacokinetics and efficacy relative to a free drug control.

12. The method according to claim 1 where the ligand design increases the solubility in water relative to the free drug control.

13. The method according to claim 12 where the bifunctional has improved oral bioavailability relative to a free drug control.

14. The method according to claim 1 where the mass of the recruiter ligand is less than 900 Daltons.

15. The method according to claim 1 where the mass of the recruiter ligand is less than 800 Daltons.

16. The method according to claim 1 where the mass of the recruiter ligand is less than 300 Daltons.

17. The method according to claim 15 where lowering the recruiter ligand mass and relative to FK506 allows improved blood-brain barrier crossing relative to a bifunctional containing FK506.

18. The method according to claim 1 where the maximum tolerated dose is at least 20% higher than the free drug control.

19. The method according to claim 1 where the maximum tolerated dose is at least 50% higher than the free drug control.

20. The method according to claim 1 where the maximum tolerated dose is at least 100% higher than the free drug control.

21. The method according to claim 1 where the bifunctional achieves equivalent efficacy to the free drug control at a concentration measured in moles/kg which is at least 33% less than the free drug control.

22. The method according to claim 1 where the bifunctional achieves equivalent efficacy to the free drug control at a concentration measured in moles/kg which is at least 66% less than the free drug control.

23. The method according to claim 1 where the bifunctional achieves equivalent efficacy to the free drug control at a concentration measured in moles/kg which is at least 80% less than the free drug control.

24. A method for improving at least one pharmacokinetic property and affinity of a therapeutic upon administration to a host, the method comprising:
administering to the host an effective amount of a bifunctional compound of less than about 5000 Daltons comprising the therapeutic or an active derivative, fragment or analog thereof and a recruiter ligand moiety,
wherein the recruiter ligand moiety binds to at least one biomoiety,
wherein the bifunctional compound has at least one modulated pharmacokinetic property upon administration to the host and equivalent or greater efficacy as compared to a free drug control that comprises the therapeutic, and
wherein the bifunctional off-rate constant, k_{offv}, has been engineered with respect to the free drug dissociation constant to produce an optimal therapeutic effect by allowing an equivalent therapeutic benefit to the free drug control at a lower concentration.

25. The method according to claim 24 where the off rate constant of the bifunctional compound from the recruited biomoiety is greater than one one-thousandth and less than 1000 times the product of the on rate constant of the free drug control to the therapeutic target multiplied by the dissociation binding constant of the free drug control to a target of the therapeutic.

26. The method according to claim 25 where the off rate constant of the bifunctional drug from the recruited biomoiety is greater than one-hundredth and less than 100 times the product of the on rate constant of the free drug control to the drug target multiplied by the dissociation binding constant of the free drug control to the target of the therapeutic.

27. The method according to claim 26 where the off rate constant of the bifunctional drug from the recruited biomoiety is greater than one tenth and less than 10 times the product of the on rate constant of the free drug control to the drug target multiplied by the dissociation binding constant of the free drug control to the target of the therapeutic.

28. A method for improving at least one pharmacokinetic property of a therapeutic upon administration to a host, the method comprising:
administering to the host an effective amount of a bifunctional compound of less than about 5000 Daltons comprising the therapeutic or an active derivative, fragment or analog thereof and a recruiter ligand moiety other than SLF,
wherein the bifunctional compound has at least one modulated pharmacokinetic property upon administration to the host as compared to a free drug control that comprises the therapeutic and the bifunctional compound has at least the same affinity of the bifunctional drug moiety to a target of the therapeutic,
wherein the intracellular vs. extra-cellular distribution of the bifunctional allows equivalent area under the curve at a bifunctional dose of no more than 50% the dose of the free drug control and
wherein the bifunctional solubility is improved relative to the same bifunctional moiety which contains SLF as the recruiter ligand.

29. The method according to claim 28, wherein the partitioning of the bifunctional compound between the extracellular and intracellular space improves pharmacokinetics and the compound exhibits equivalent in vivo efficacy at a bifunctional dose of no more than 33% of the free drug control.

30. The method according to claim 28, wherein the partitioning of the bifunctional compound between the extracellular and intracellular space improves pharmacokinetics and the compound exhibits equivalent in vivo efficacy at a bifunctional dose of no more than 20% of the free drug control.

31. The method of claim 1, wherein at least one intracellular protein bound comprises an FK506 binding protein, tubulin, actin, a heat shock protein, or albumin.

32. The method according to claim 1, where the in vivo efficacy of the bifunctional compound in the presence of a suitable protein to which the recruiter ligand moiety couples is increased by a factor of at least about 2 relative to the in vivo efficacy of equimolar free drug due to equivalent or improved affinity as well as improved pharmacokinetics due to the presence of the recruiter ligand moiety and recruited biomoiety.

33. The method according to claim 1, where the in vivo efficacy of the bifunctional compound in the presence of a suitable protein to which the recruiter ligand moiety couples is increased by a factor of at least about 4 relative to the in vivo efficacy of equimolar free drug due to equivalent or improved affinity as well as improved pharmacokinetics due to the presence of the recruiter ligand moiety and recruited biomoiety.

34. The method according to claim 1, where the in vivo efficacy of the bifunctional compound in the presence of a suitable protein to which the recruiter ligand moiety couples is increased by a factor of at least about 8 relative to the in vivo efficacy of equimolar free drug due to equivalent or improved affinity as well as improved pharmacokinetics due to the presence of the recruiter ligand moiety and recruited biomoiety.

35. The method of claim 1 where the partitioning of the bifunctional compound from the extracellular to the intracellular space is changed by at least factor of 20% relative to the free drug control.

36. The method of claim 1 where the partitioning of the bifunctional compound from the extracellular to the intracellular space is changed by at least factor of 40% relative to the free drug control.

37. The method of claim 1 where the partitioning of the bifunctional compound from the intracellular to the extracellular space is changed by at least factor of 20% relative to the free drug control.

38. The method of claim 1 where the partitioning of the bifunctional compound from the intracellular to the extracellular space is changed by at least factor of 40% relative to the free drug control.

39. The method of claim 1 where the affinity of the bifunctional for the recruited biomoiety is substantially equivalent to the affinity of the bifunctional to a drug efflux mechanism protein.

40. The method of claim 1 where the affinity of the bifunctional for the recruited biomoiety is more than twice affinity of the bifunctional to a drug efflux mechanism protein.

41. The method of claim 1 where the affinity of the bifunctional for the recruited biomoiety is more than three times the affinity of the bifunctional to a drug efflux mechanism protein.

42. The method of claim 1 where the ligand binds to a peptide which binds to the epidermal growth factor receptor.

43. The method of claim 1 where the pharmacokinetics and affinity are optimized by varying the attachment point of the linker to the drug moiety in a bifunctional drug.
